(19) United States
(12) Patent Application Publication
(10) Pub. No.: US 2007/0088090 A1
(43) Pub. Date: Apr. 19, 2007

(54) Alpha-bulnesene, its preparation and its use

(75) Inventors: Hui-Chun Hsu, Taipei (TW); Wen-Chia Yang, Taipei (TW); Ying-Chieh Tsai, Taipei (TW)

Correspondence Address: BACON & THOMAS, PLLC, 625 SLATERS LANE, FOURTH FLOOR, ALEXANDRIA, VA, 22314, US

(73) Assignee: Yangsen Biotechnology Co., Ltd., Taipei (TW)

(21) Appl. No.: 11/249,475

(22) Filed: Oct. 14, 2005

(51) Int. Cl. A61K 31/015; C07C 13/615

(52) U.S. Cl. 514/763; 585/22

(57) ABSTRACT

The present invention discloses α-bulnesene isolated from the essential oil of the plant of Pogostemon cablin. Steam distilled essential oil from the aerial parts of Pogostemon cablin was isolated to obtain α-Bulnesene. The invented α-Bulnesene inhibited platelet aggregation induced by platelet-activating factor (PAF) or arachidonic acid (AA). Use of the invented α-bulnesene as anti-inflammation agent is disclosed. The invented α-Bulnesene has the following chemical structure: 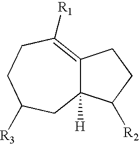 wherein R1, R2 and R3 are respectively H, C51-C4 alkyl or isopropyl.

### FIELD OF THE INVENTION

The present invention relates to α-bulnesene, its preparation and its use, especially to α-bulnesene prepared from the essential oil of Pogostemon cablin, and its usage as anti-inflammation agent, in particularly as anti-PAF agent.

### BACKGROUND OF THE INVENTION

Platelet aggregation plays an important role in thrombosis and hemostasis. The first step in the response of platelets to vascular injury is irreversible attachment to the altered surface followed by platelet activation. This mainly takes place by the action of certain agonists such as platelet-activating factor (PAF), arachidonic acid (AA), etc. When platelets are activated by these aggregation agonists, they undergo shape changes, secretary process and subsequently express fibrinogen receptors on their surface. The fibrinogen binding to its specific receptors associated with glycoprotein (GP) IIb/IIIa complex appears to be a prerequisite for the formation of platelet plug. However, numerous evidences indicated that platelets contribute significantly to the etiology and pathogenesis of acute coronary syndrome, myocardial infraction and embolismic stroke, which are the major causes of death in developed countries.

The activation of platelets can be counter-regulated by processes that attenuate or prevent agonist-induced responses. Anti-platelet agents inhibiting platelet activation by certain mechanisms, such as aspirin, a nonspecific cyclooxygenase inhibitor which dampens the production of thromboxane A₂ (TXA₂), have been shown to be clinically beneficial in the treatment of thromboembotic diseases. However, current anti-platelet drugs still have considerable limitation in their mode of action and efficacy. Greater understanding of platelet function in molecular levels will probably lead to the development of novel anti-platelet drugs.

Pogostemon cablin (Labiatae) is the aerial part of P. cabin Bentham and has been used for the treatment of common cold and as an antifungal agent in Chinese traditional medicine. This plant is important to the perfumery industry and is cultivated extensively in Indonesia, Malaysia, China, and Brazil for its essential oil (patchouli oil). A number of investigations has been carried out on the composition of water extract and essential oil from P. cablin, and the presence of a number of mono- and sesquiterpenoids has been reported. In addition, several flavonoids and alkaloids have been isolated and identified from P. cablin. Although the chemical constituents of P. cablin have frequently been investigated, there have been no reports concerning any bioactive compound extracted from the essential oil of P. cablin.

The bioactivity-guided fractionation of extract from plants has been executed frequently to find active compounds from within the plants. Various plant-derived essential oils have been traditionally and experimentally proved to have bioactivity, and many studies have tried to identify the main active compounds. Recent studies have shown that sesquiterpenes may inhibit mutagenesis induced by chemical mutagens. In addition, a large variety of pharmacological activities has been reported, for example, anticarcinogenic activity and antioxdative activity. However, inhibition of PAF-induced platelet aggregation by sesquiterpenes has not been reported previously.

In U.S. patent application Ser. No. 10/880,651 a herbal composition including essential oil of P. cablin in the use of inhibition or prevention of allergic syndromes were disclosed. The composition comprises essential oils of Houxiang berba, Qianghuo herba and Xinyi herba. According to the invention, anti-allergic effects of the composition were proved to be promising. However, effective chemical ingredients in the composition was not disclosed.

### OBJECTIVES OF THE INVENTION

The objective of this invention is to isolate effective chemical ingredients for inhibition and prevention of allergic syndromes from essential oils of herbal medicines.

Another objective of this invention is to provide a new anti-inflammation medicine that may be isolated from essential oils of herbal medicines.

Another objective of this invention is to provide a new method for the preparation of anti-allergic medicines.

Another objective of this invention is to provide a new anti-allergic medicine that exhibits fewer side effects.

Another objective of this invention is to provide a new chemical compound that inhibits allergic agonists such as platelet-activating factor (PAF) and arachidonic acid (AA).

### SUMMARY OF THE INVENTION

According to the present invention, α-bulnesene isolated from the essential oil of Pogostemon cablin is disclosed. The α-bulnesene of this invention has the chemical structure as follows:

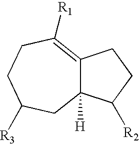

wherein R1, R2 and R3 are respectively H, C1-C4 alkyl or isopropyl.

Essential oils of P. cablin are prepared and isolated to obtain major ingredients. Selected ingredients are further isolated to obtain α-bulnesene compositions. The obtained α-bulnesene exhibited significant inhibitory activity against platelet aggregation induced by Platelet Activating Factor (PAF), an inflammatory phospholipid mediator produced by various cells and involved in allergic disease, inflammation, asthma, rhinitis, shock, and cardiovascular disease. Anti-allergic compounds are thus obtained.

These and other objectives and advantages of this invention may be clearly understood from the following detailed description of the invention.

### BRIEF DESCRIPTION OF THE DRAWINGS

FIG. 1 shows the gas chromatography of the essential oil of Pogostemon cablin generated by thermal conductivity detector.

FIG. 2 shows the inhibitory effects of α-bulnesene against platelet aggregation induced by PAF and AA.

FIG. 3 shows inhibitory effects of α-bulnesene in competition to PAF receptor binding.

FIG. 4 shows the effect of α-bulnesene on PAF-induced Ca2+ mobilization, wherein FIG. 4a shows the case where Fluo-3-loaded platelet was pretreated with isopropanol (0.5%), FIG. 4b shows the case where α-bulnesene (50 μM) was added and FIG. 4c shows the percentage of Ca2+ release under different concentrations of α-bulnesene induced by PAF

Table I shows the inhibitory effects of fractions 1˜4 of the essential oil of Pogostemon cablin against PAF-induced platelet aggregation.

Table II shows effects of α-bulnesene on TXB2 level induced by AA in platelets.

### DETAILED DESCRIPTION OF THE INVENTION

General Descriptions

In the present invention, an anti-allergic agent isolated from the essential oil of Pogostemon cablin is disclosed. The anti-allergic agent is identified as α-bulnesene and has the following chemical structure:

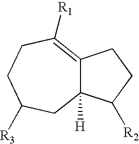

wherein R1, R2 and R3 are respectively H, C1-C4 alkyl or isopropyl.

The invented α-bulnesene expresses significant inhibitory activity against platelet aggregation induced by Platelet Activating Factor (PAF) and may be used as effective ingredient for anti-allergic medicines.

Essential oil of P. cablin is obtained using conventional method of extracting technology, including steam distilation. The essential oil was analyzed on a conventional gas-chromatograph with a flame ionization detector. The oven temperature was programmed to isolate major ingredients from the essential oil. The inhibitory effects of the isolated fractions were tested and the fraction with remarkable effected was selected and subjected to further isolation. All the subfractions were collected and tested, whereby the subfraction with best inhibitory effects was selected for purification. The mass spectra of the products were recorded on a selective quadrupolar type detector. Ionization was obtained using electron impact. Components were identified according to their GC retention times and retention indices. Results are compared with available library using a computer to find their mates. The produced fragmentation patterns were also compared with those reported in the previous literatures or even co-injected with known samples.

The assays were prepared and commercially available potent PAF antagonist was used as positive reference compound. Blood samples were collected from the marginal ear artery of New Zealand white rabbits. The blood was then treated to obtained platelet-rich plasma (PRP) mixed with EDTA. The platelet pellet was obtained from the EDTA-anticoagulated PRP by centrifugation. The pellet was then suspended in Tyrode solution and incubated. Platelet numbers were then counted.

The platelet suspension was stirred with solvent of test compound in solvent of different concentrations. Aggregation inducer PAF was then added to trigger the aggregation. Aggregation was determined by the transmission method using a platelet aggregation chromogenic kinetic system. The initial transmittance of the Ca²⁺-containing Tyrode solution was taken as 0% aggregation and that of the platelet suspension as 100% aggregation. The IC₅₀ value was defined as the concentration of the sample that inhibited 50% of the aggregation. Percent inhibition by a compound was calculated.

Isolation of α-bulnesene

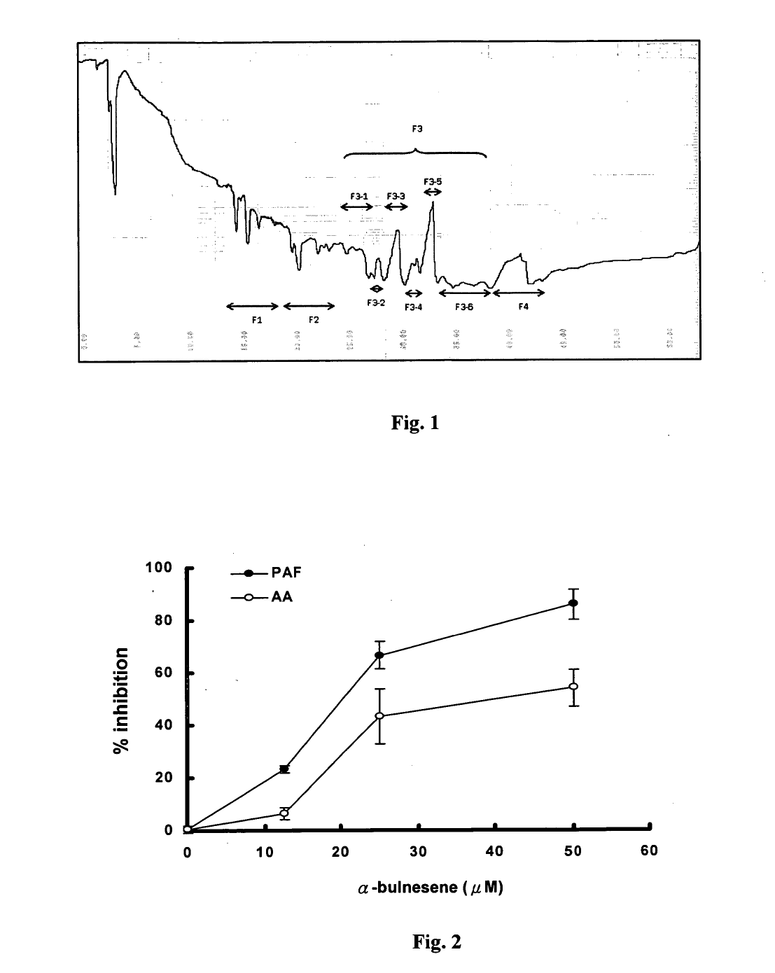

Commercially available steam distilled essential oils of Pogostemon cablin were obtained. To purify the active principles, the essential oil of P. cablin was subjected to chromatographic procedures and the fractions monitored by the platelet aggregation assay. FIG. 1 shows the gas chromatography of the essential oil of Pogostemon cablin generated by thermal conductivity detector. As shown in this figure, the chromatographic procedure used for fractionation and identification of the suppressive compound by GC-TCD was performed on a Hewlett-Packard 5890 gas chromatograph equipped with a thermal conductivity detector. The oven temperature was held at 50° C. for 4 min, then programmed from 50 to 272° C. at 6° C./min, and finally held at 272° C. for 3 min. Four fractions F1 (retention time 15-23 min), F2 (24-29 min), F3 (30-39 min), and F4 (40-50 min) were obtained. Among them F3 showed inhibitory activity against PAF-induced platelet aggregation. Table I shows the inhibitory effects of fractions 1˜4 of the essential oil of Pogostemon cablin on PAF-induced platelet aggregation.

The F3 composition was collected. Modify the chromatographic procedure to divide F3 into more detailed sections. The oven temperature was held at 50° C. for 4 min, then programmed from 50 to 170° C. at 10° C./min, 170 to 272° C. at 3° C./min, and finally held at 272° C. for 3 min.

F3 was further divided and collected to six sub-fractions F3-1 (retention time 24-27 min), F3-2 (27-28 min), F3-3 (28-30 min), F3-4 (30-31.5 min), F3-5 (31.5-33 min), and F3-6 (33-38 min), as shown in FIG. 1. Sub-fraction F3-5 showed the strongest inhibitory activity against PAF-induced platelet aggregation. See Table I. Table I shows the inhibitory effects of fractions 1˜4 of the essential oil of Pogostemon cablin against PAF-induced platelet aggregation.

The suppressive compound in F3-5 was isolated. From the spectral data and its physical properties, it was identified as α-bulnesene, a sesquiterpene produced by sesquiterpene cyclase patchoulol synthase in P. cablin, having the following chemical structure:

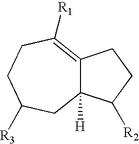

wherein R1, R2 and R3 are respectively H, C1-C4 alkyl or isopropyl.

The chemical is further analyzed and identified as 1β,4-dimethyl-7β-isopropenyl-1,2,3,5,6,7,8,8aα-octahydroazulene[α-bulnesene] by MS, and ¹H, and ¹³C NMR spectroscopes. The compound is a yellow liquid and exhibited the expected spectral properties: MS, m/z 316 (M⁺ 75), 180 (60), 167 (100), 150 (37), 137 (71); IR spectrum, _{γmax} KBr (cm⁻¹) 3417, 1645, 1575, 1519, 1269, 1158; λmax 3.50, 5.76μ; NMR spectrum, two broad singlets at τ 5.36-5.62 (2H, exocyclic methylene), and two near-singlets at τ 8.37 (6H, two methyl groups attached to double bonds), which all supported the structure of α-bulnesene well.

Allergy Inhibitory Effects of α-Bulnesene

α-Bulnesene was dissolved in isopropanol. Platelet-activating factor (PAF), arachidonic acid (AA), fluo-3/AM, indomethacin, ethylenediaminetetraacetic acid (EDTA, disodium salt) and trichloroacetic acid (TCA) were purchased from Sigma Chemical (St Louis, Mo.). Thromboxane B₂ and Prostaglandin E₂ enzyme immunoassay kits were obtained from Cayman Chemical Co. (Ann Arbor, Mich.). [³H] PAF (1-O-[³H]octadecyl-2-acetyl-sn-glycero-3-phosphocholine), with a specific radioactivity of 120 Ci mmol⁻¹, a product of PerkinElmer Science, was dissolved in absolute ethanol and diluted with saline solution containing 2.5 mg mL⁻¹ bovine serum albumin (BSA) immediately prior to use. All other chemicals were of the highest purity grade available.

Platelet suspension was prepared as follows. Blood was collected from the marginal ear vein of New Zealand White rabbits. The blood was then anticoagulated with acid citrate dextrose (ACD, 1:6, v/v) and centrifuged for 8 min at 1000×g at room temperature. The upper portion was collected as platelet-rich plasma (PRP) and mixed with EDTA to a final concentration of 5 mM. The platelet pellets were obtained from EDTA-mixed PRP by centrifugation for 12 min at 2000×g. The platelet pellets were suspended with Ca²⁺-free Tyrode's solution, and then incubated with apyrase (1 unit mL⁻¹) for 15 min at 37° C. After centrifugation for 6 min at 2000×g, the platelet pellets were finally suspended in Tyrode's solution of the following composition (mM): NaCl (137), KCl (2.8), MgCl₂ (2), NaH₂PO₄ (0.33), CaCl₂ (1), glucose (5) and HEPES (10) containing BSA (0.35%), pH 7.3. The concentration of platelets was adjusted to 2.5×10⁸ platelets mL⁻¹.

Effects of α-Bulnesene on Platelet Aggregation

Platelet aggregation was measured turbidimetrically with a light-transmission Platelet Aggregation Chromogenic Kinetic System PACK4 (Helena Laboratories, Beaumont, Tex., USA) with some modifications. Platelet suspension stirred at 900 rev min⁻¹ was incubated with isopropanol (solvent) or various concentrations of α-bulnesene at 37° C. for 2 min. Aggregation was induced with PAF (5 nM) and AA (100 μM). The absorbance of the Tyrode's solution was taken as 0% aggregation and that of the platelet suspension as 100% aggregation. The extent of platelet aggregation was measured as the maximal increase of light transmission within 4 min after addition of the inducers. When isopropanol was used as the solvent the final concentration was fixed at 0.5% (v/v) to eliminate the effect of the solvent. All glassware was siliconized.

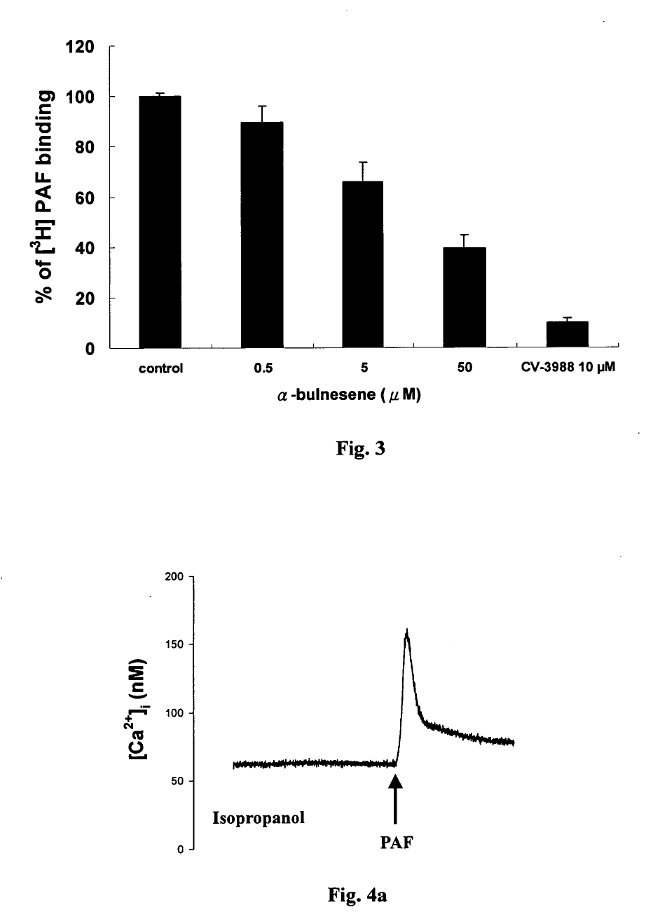

The inhibitory effect of α-bulnesene on PAF- and AA-induced platelet aggregation was determined. FIG. 2 shows the inhibitory effects of α-bulnesene against platelet aggregation induced by PAF (5 nM) and AA (100 μM). PAF (5 nM) and AA (100 μM) both caused about 70-80% aggregation in rabbit washed platelets. As shown in FIG. 2, α-Bulnesene (12.5-50 μM) markedly inhibited platelet aggregation stimulated by PAF and AA in a concentration-dependent manner with IC₅₀ values of 24.47±2.45 μM and 42.47±5.97 μM, respectively. In contrast, CV-3988, the specific PAF receptor antagonist, almost completely inhibited PAF-induced platelet aggregation with the IC₅₀ value of 2.40±1.01 μM. It however showed no inhibition on AA-induced platelet aggregation.

Effect of α-Bulnesene on [³H]PAF Binding of Platelets

[³H]PAF receptor binding assay is prepared using rabbit washed platelets. The reaction mixture consisted of 400 μl of platelet suspension, 50 μl of [³H]PAF (0.2 nM, 100,000 dpm) with or without unlabeled PAF (1.5 μM) and 50 μl of sample or control solution. The reaction mixture was incubated at 4° C. for 2 hrs and then 5 mL of BSA-containing saline solution was added to terminate the reactions. The free and bound ligands were separated by filtration using a Whtman GF/C glass fiber filters which were presoaked with ice-cold water. The filters were rapidly washed with the same solution and then dried and placed into vials containing 10 mL of the scintillation fluid. Radioactivity was measured in a liquid scintillation counter (Beckman LS3801). The difference between the total radioactivity of bound [³H]PAF in the absence and in the presence of excess unlabeled PAF is defined as specific binding of the radio-labeled ligand. In a batch of experiments, [³H]PAF was incubated with different concentrations of PAF receptor antagonists, and the effect of the antagonist on the specific binding was expressed as percentage inhibition of the control. The IC₅₀ value was defined as the final concentration of the inhibitor required to block 50% of the specific [³H]PAF binding to rabbit platelet receptors, as follows:

Percent inhibition (%)=[100−(B/A×100)](%)

wherein A represents percent aggregation in control added and B represents percent aggregation in compound added.

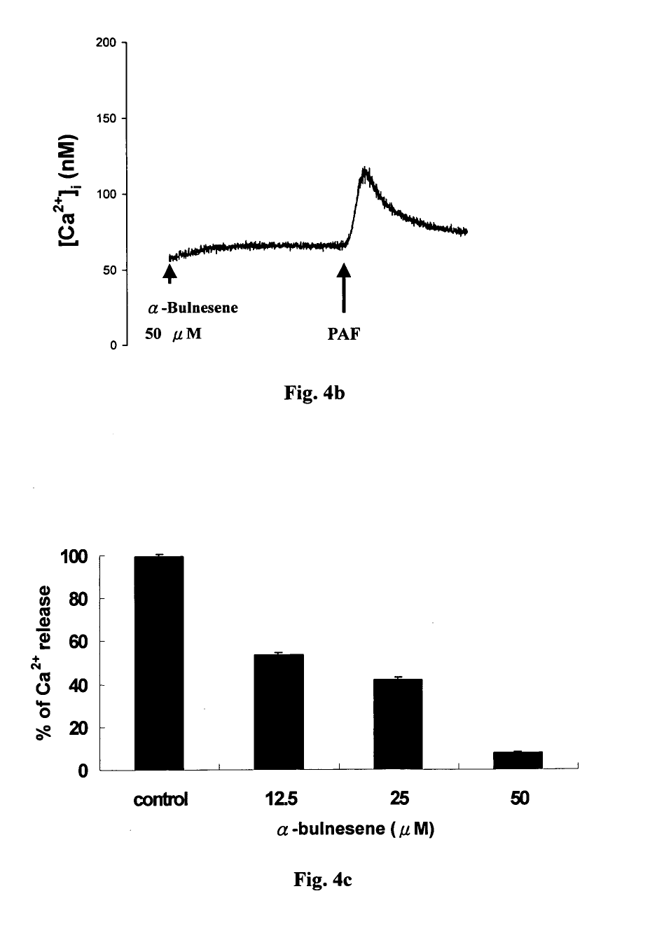

The total binding of 0.2 nM [³H]PAF to intact rabbit platelets (1.3×10⁸ platelets mL⁻¹) in the presence of BSA (2.5 mg mL⁻¹) was 109,131.17±4,534.02 dpm (n=6), whereas that of non-specific binding in the presence of 1.5 μM unlabeled PAF was 2,339.03±292.18 dpm. FIG. 3 shows inhibitory effects of α-bulnesene in competition to PAF receptor binding. As shown in FIG. 3, α-bulnesene concentration-dependently inhibited [³H]PAF binding to rabbit platelets. The IC₅₀ value of α-bulnesene for [³H]PAF binding was 17.62±5.68 μM. On the other hand, the percent inhibition of CV-3988 (10 μM) for [³H]PAF binding was 10.10±1.74% (n=3).

Effect of α-Bulnesene on the Intracellular Calcium Release of Platelets

Platelets pelleted from PRP were resuspended in Ca²⁺-free Tyrode's solution, and then incubated with fluo-3/AM (2 μM) at 37° C. for 30 min. In order to prevent leakage of dye, probenecid (2.5 mM) was added to the buffers throughout the experiments. After washing, the fluo-3-loaded platelets were finally suspended in Ca²⁺-free Tyrode's solution without heparin at a concentration of 1.3×10⁸ platelets mL⁻¹. The fluo-3-loaded platelets were preincubated with α-bulnesene in the presence of extracellular calcium (1 mM) at 37° C. for 2 min prior to addition of PAF. Fluorescence (Ex 505 nm, Em 530 nm) was measured with a fluorescence spectrophotometer (Model F4500; Hitachi, Tokyo, Japan). At the end of the experiment, the cells were treated with digitonin (20%) followed by the addition of EGTA (50 mM) to obtain the maximal and minimal fluorescence, respectively. [Ca²⁺]ᵢ was calculated as described for fluo-3/AM using the Ca²⁺-dye dissociation constant 864 nM.

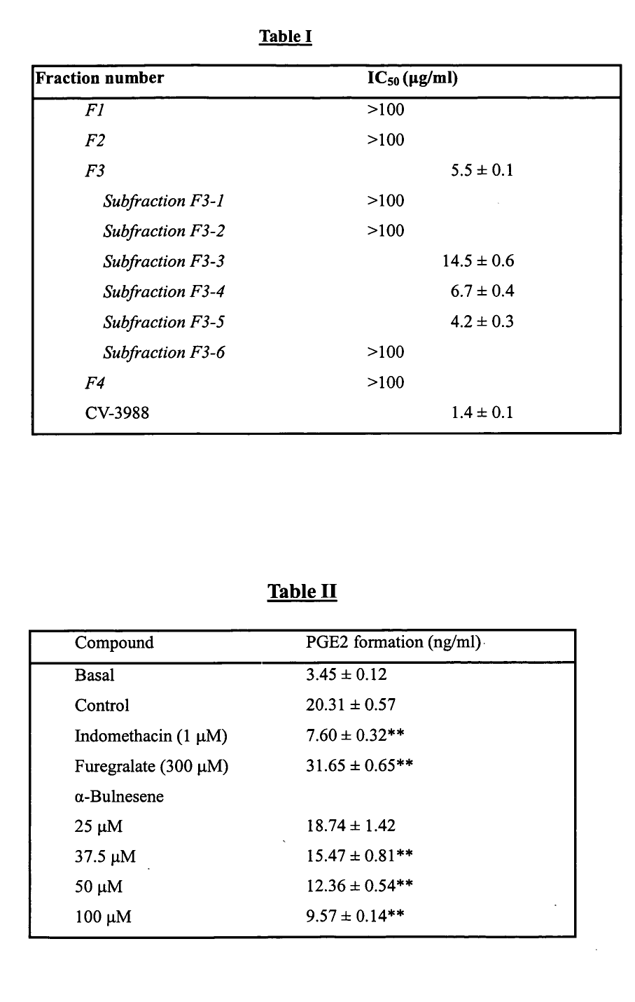

FIG. 4 shows the effect of α-bulnesene on PAF-induced Ca2+ mobilization, wherein FIG. 4a shows the case where Fluo-3-loaded platelet was pretreated with isopropanol (0.5%) and FIG. 4b shows the case where α-bulnesene (50 μM) was added. FIG. 4c shows the percentage of Ca2+ release under different concentrations of α-bulnesene induced by PAF. As shown in these figures, in fluo-3-loaded platelets, the resting level of intracellular Ca²⁺ concentration was 61.80±1.04 nM, and PAF (5 nM) causes an increase of the concentration to 167.01±5.17 nM (FIG. 4a). α-Bulnesene (2.5-50 μM) inhibited PAF-induced intracellular Ca²⁺ release in a concentration dependent manner with IC₅₀ value of 19.62±1.32 μM, as shown in FIG. 4b.

Effect of α-Bulnesene on Thromboxane B₂ and Prostaglandin E₂ Formation

Because TXA₂ is very unstable and may be rapidly converted to more stable metabolite TXB₂, in this experiment the amount of TXB₂ was measured. After challenging platelets with AA (100 μM) for 6 min, 1 mM EDTA and 20 μM indomethacin were added to terminate the reaction. By centrifugation for 5 min at 4° C. and 10,000×g, the TXB₂ and PGE₂ in the supernatants were assayed using EIA kits according to the procedure described by the manufacturer.

Thromboxane B₂ and prostaglandin E₂ formation in rabbit washed platelets was measured at 6 min after AA was added. Table II shows effects of α-bulnesene on TXB2 level induced by AA in platelets. As shown in Table II, the resting level of TXB₂ in rabbit platelets was 7.64±0.40 ng mL⁻¹. AA (100 μM) markedly increased TXB₂ formation to 568.51±42.63 ng mL⁻¹. TXB₂ formation stimulated by AA was inhibited by α-bulnesene (25-100 μM) in a concentration-dependent manner. Indomethacin (1 μM) and furegrelate (300 μM) were chosen as positive control, with percent inhibition of 98.05±0.16% and 96.49±0.78%, respectively. On the other hand, PGE₂ was also formed in the presence of AA. PGE₂ formation in rabbit platelet suspension was 3.45±0.12 ng mL⁻¹ in the unstimulated condition, while AA raised the PGE₂ level to 20.31±0.57 ng mL⁻¹. This formation was inhibited by α-bulnesene (25-100 μM). PGE₂ formation was also inhibited by indomethacin and was reversely enhanced by furegrelate (300 μM).

### EFFECTS OF THE INVENTION

As described above, the invented α-bulnesene inhibited platelet aggregation induced by PAF and arachidonic acid, with the IC₅₀ values of 24.47±2.45 μM and 42.47±5.97 μM, respectively. In the cytotoxicity assay, α-bulnesene (12.5-50 μM) did not have any toxicity in rabbit platelets even after a 30 min treatment (data not shown). This indicated that the inhibition of α-bulnesene on platelet aggregation did not go through cytotoxicity.

In conclusion, α-bulnesene is a novel sesquiterpenoids referred as PAF receptor competitive inhibitor. Moreover, α-bulnesene also possessed the additional inhibitory effect on exogenous AA-induced secondary platelet aggregation and TXA₂ formation through affecting the COX activity.

What is claimed is:

1. α-Bulnesene having the following chemical structure: 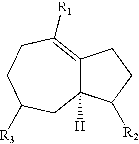
wherein R1, R2 and R3 are respectively H, C1-C4 alkyl or isopropyl.

2. 1β,4-dimethyl-7β-isopropyl-1,2,3,5,6,7,8,8aα-octahydroazulene.

3. Chemical exhibiting the spectral properties of: MS, m/z 316 (M⁺ 75), 180 (60), 167 (100), 150 (37), 137 (71); IR spectrum, _{γmax} KBr (cm⁻¹) 3417, 1645, 1575, 1519, 1269, 1158; λmax 3.50, 5.76μ; NMR spectrum, two broad singlets at τ 5.36-5.62 (2H, exocyclic methylene), and two near-singlets at τ 8.37.

4. Method for preparation of α-bulnesene, comprising the following steps:
extracting essential oil from plant comprising Pogostemon cablin;
dividing said essential oil chromatographically into fractions;
selecting from said fractions fraction showing inhibitory activity against platelet aggregation; and
isolating α-bulnesene from said selected sub-fraction.

5. The method according to claim 4, wherein said platelet aggregation includes PAF-induced platelet aggregation.

6. The method according to claim 4, wherein said platelet aggregation includes arachidonic acid-induced platelet aggregation.

7. The method according to claim 4, wherein said α-bulnesene has the chemical structure of: 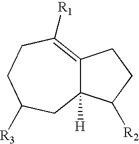
wherein R1, R2 and R3 are respectively H, C1-C4 alkyl or isopropyl.

8. The method according to claim 4, wherein said α-bulnesene is 1β,4-dimethyl-7β-isopropyl-1,2,3,4,5,6,7,8,8aα-octahydroazulene.

9. Use of α-bulnesene as anti-inflammation agent.

10. The use according to claim 9, wherein said said α-bulnesene has the following chemical structure: 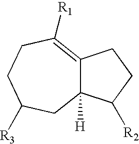
wherein R1, R2 and R3 are respectively H, C1-C4 alkyl or isopropyl.

11. The use according to claim 9, wherein said α-bulnesene is 1β,4-dimethyl-7β-isopropyl-1,2,3,5,6,7,8,8aα-octahydroazulene.

12. The use according to claim 9, wherein said α-bulnesene is prepared using the following steps:
extracting essential oil from plant comprising Pogostemon cablin;
dividing said essential oil chromatographically into fractions;
selecting from said fractions fraction showing inhibitory activity against platelet aggregation; and
isolating α-bulnesene from said selected sub-fraction.

13. The use according to claim 12, wherein said platelet aggregation includes PAF-induced platelet aggregation.

14. The use according to claim 12, wherein said platelet aggregation includes arachidonic acid-induced platelet aggregation.
